(19) United States
(12) Patent Application Publication
(10) Pub. No.: US 2022/0257662 A1
(43) Pub. Date: Aug. 18, 2022

(54) NERVOUS SYSTEM CELL THERAPY

(71) Applicant: TESSARA THERAPEUTICS PTY LTD, Melbourne (AU)

(72) Inventors: Christopher BOYER, Blackburn (AU); Christos PAPADIMITRIOU, Blackburn (AU)

(73) Assignee: Tessara Therapeutics Pty Ltd, Melbourne (AU)

(21) Appl. No.: 17/309,113

(22) PCT Filed: Oct. 25, 2019
(86) PCT No.: PCT/AU2019/051174

§ 371 Date: Apr. 26, 2021

(30) Foreign Application Priority Data
Oct. 26, 2018 (AU) 2018904068

(51) Int. Cl. A61K 35/30; A61K 9/06; A61K 9/50; A61K 9/00

(52) U.S. Cl. CPC A61K 35/30; A61K 9/06; A61K 9/5057; A61K 9/5042; A61K 9/5036; A61K 9/0024; A61K 9/0085; A61K 9/5031

(57) ABSTRACT

The present invention relates to methods for regenerating nervous system tissue or treating a neurological disorder by administration of a therapeutically effective amount of synthetic tissue containing a cell population of one or more nervous system cell types (e.g., neurons) or multipotent cells (e.g., mesenchymal stem cells), where the cell population is embedded within a modular synthetic hydrogel that is biocompatible. In some preferred embodiments the modular synthetic hydrogel includes a PEG hydrogel crosslinked with a glycosaminoglycan such as hyaluronan.

### CROSS-REFERENCE TO RELATED APPLICATIONS

This application is a national phase application under 35 U.S.C. § 371 of International Application No. PCT/AU2019/051174 filed Oct. 30, 2019, which claims the benefit of priority of Australian Patent Application No. 2018904068 filed Oct. 26, 2018, all of which are hereby incorporated by reference in their entirety.

### FIELD

The specification relates generally to the field of cell therapy. More particularly, the specification relates to methods for generating human neural tissue-mimetic constructs to regenerate neural tissue in vivo and/or to treat neurological conditions.

### BACKGROUND

Neurological disorders present a massive health burden. Neurodegenerative diseases such as Alzheimer's, Parkinson's, and Huntington's disease, as well as stroke are becoming ever more prevalent in our society. In fact, stroke is the leading cause of adult disability in developed countries. Many neurological disorders and diseases are associated with a loss of neurons resulting in a deficiency of specific neurotransmitters, e.g., the loss of midbrain dopaminergic neurons in Parkinson's disease.

While a pharmacological approach to restoring neurotransmitter levels has been employed for many years (e.g., the use of L-DOPA in Parkinson's), this therapeutic avenue has a number of well known disadvantages. For example, it is very challenging to establish a therapeutically effective drug dosage in patients, as the therapeutic window actually narrows over time. Indeed, ultimately, such drugs become ineffective. Thus, there is a longstanding need for improved methods to effectively treat neurotransmitter deficits and loss of function in neurological disorders. A relatively new approach, cell therapy, is based on the administration of disease-relevant cell types to restore neurotransmission and function, e.g., fetal dopaminergic neuron transplantation to treat Parkinson's. However, while this is a promising approach, success to date has been limited by a number of factors including the relatively poor survival of transplanted cells, lack of maturation and/or connectivity of the donor cells, and poor localisation of the donor cells at the required sites within the host neural tissue. Thus, there is an ongoing need for improved cell therapy methods for treating neurological disorders that address these shortcomings.

### SUMMARY

The present invention relates to repair of nervous system tissue and/or treatment of neurological disorders by administration of tissue-mimetic constructs based on 3D culture and maturation of one or more cell types embedded within a biocompatible, modular synthetic hydrogel.

Accordingly, in one aspect the present invention provides a method for repairing nervous system tissue in a subject in need thereof, comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible.

In another aspect, the present invention provides a method for treating a neurological disorder in a subject in need thereof, comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible.

In a further aspect, the present invention provides a method for repairing nervous system tissue, or for treating a neurological disorder in a subject in need thereof comprising administering to the subject a therapeutically effective amount of a synthetic tissue obtained by a process comprising the steps of :
(i) embedding a cell population comprising (a) one or more nervous system cell types, or (b) multipotent cells in a modular synthetic hydrogel; and
(ii) culturing the embedded cell population for a period.

In one embodiment, the subject is suffering from a neurological disorder selected from the group consisting of: Alzheimer's Disease, vascular dementia, Parkinson's Disease, Huntington's Disease, stroke, ischemic stroke, haemorrhagic stroke, optic nerve disease, spinal cord injury, peripheral nerve injury, demyelinating disease, and traumatic brain injury. In some embodiments the subject to be treated is suffering from a surgical wound.

In one embodiment, the multipotent cells are mesenchymal stem cells.

In one embodiment, the nervous system cell types are selected from the group consisting of neurons, neural progenitor cells, glial cells, and any combination thereof.

In one embodiment, the cell population comprises neurons.

In one embodiment, the neurons are selected from the group consisting of monoaminergic neurons, catecholaminergic neurons, glutamatergic excitatory neurons, GABAergic inhibitory neurons, motor neurons, cholinergic neurons, and any combination thereof.

In one embodiment the cell population comprises dopaminergic neurons. In one embodiment, the cell population comprises neurones or domaminergic neurones that are excitatory and inhibitory neurons in a predetermined ratio.

In another embodiment, the cell population comprises glial cells.

In one embodiment, the glial cells are astrocytes.

In another embodiment, the glial cells are myelinating glial cells or microglia.

In one embodiment, the specification enables a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, and wherein the population of cells comprises neurons and glial cells.

In a further embodiment, the specifications provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the population of cells further comprises endothelial cells.

In one embodiment, the cells are immature or have not undergone incubation or maturation prior to administration.

In one embodiment, the modular synthetic hydrogel comprising the cell population is administered without an incubation or maturation step.

In one embodiment, the specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein neurons in the cell population prior to the administration exhibit at least one functional characteristic associated with neuronal maturation selected from the group consisting of: secretion of a cognate neurotransmitter, secretion of a growth factor, expression of a mature neuronal protein marker, surface expression or subcellular localisation of a neurotransmitter receptor, intrinsic electrical activity, and synaptic connectivity.

In one embodiment, both excitatory and inhibitory neurotransmission is exhibited in the cell population prior to the administration.

In one embodiment of the method, neurons in the cell population secrete a cognate neurotransmitter.

In one embodiment, the cognate neurotransmitter is selected from the group consisting of dopamine, acetylcholine, or serotonin.

In another embodiment, the specification provides a method as described herein, comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the cell population comprises genetically modified cells.

In one embodiment, the genetically modified cells express one or more exogenous proteins selected from the group consisting of: light-sensitive ion channels, chemogenetically engineered proteins, reporter proteins, optogenetic probe proteins, growth factors, transcription factors, antibodies, and cytokines.

In another embodiment, the specification provides a method as described herein, comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the cell population comprises allogeneic cells.

In another embodiment, the cell population comprises autogeneic cells.

In another embodiment, the cell population comprises both allogeneic and autogeneic cells.

In one embodiment, the cell population is distributed or embedded non-uniformly within the modular synthetic hydrogel.

In one embodiment, the cell population has a predetermined spatial distribution within the modular synthetic hydrogel.

In one embodiment of the method or use, different cell types in the predetermined spatial distribution are organised into separate layers.

In another embodiment, the present specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, and wherein the modular synthetic hydrogel comprises one or more hydrogel subunit materials.

In one embodiment, the one or more hydrogel subunit materials is/are selected from the group consisting of polyethylene glycol (PEG), hyaluronan (HAL), thiol-modified hyaluronan, acrylated hyaluronic acid thiol-modified chondroitin sulfate, gelatin, thiol-modified gelatin, collagen (COL), acrylic copolymers, polyvinylidene fluoride, chitosan, polyurethane isocyanates, polyalginate, cellulose acetate, polysulfone, polyvinyl alcohols (PVA), and polyacrylonitrile.

In one embodiment, the one or more hydrogel subunit materials comprises polyethylene glycol (PEG).

In one embodiment, the modular synthetic hydrogel further comprises one or more peptides or polypeptides linked to the one or more hydrogel subunit materials.

In one embodiment, the one or more peptides or polypeptides comprise at least a first and a second peptide or polypeptide.

In one embodiment, at least one of the one or more linked peptides or polypeptides is enzymatically cleavable.

In one illustrative embodiment, the enzymatically cleavable peptide or polypeptide comprises a metalloprotease cleavage site.

In one embodiment, at least one of the one or more linked peptides or polypeptides comprises a cell binding sequence.

In one embodiment, at least one of the one or more linked peptides comprises a convertible functional group.

In one embodiment, the convertible functional group is a maleimide group or a thiol group.

In one embodiment, the one or more peptides or polypeptides comprise heparin or a heparin derivative.

In one embodiment, the one or more peptides or polypeptides comprise a thiol-modified heparin

In one embodiment of the method, the modular synthetic hydrogel comprises PEG and at least one of heparin, hyaluronan, and collagen.

In one embodiment, the modular synthetic hydrogel comprises PEG and heparin.

In one embodiment, the modular synthetic hydrogel comprises PEG and hyaluronan.

In one embodiment, the modular synthetic hydrogel comprises PEG and collagen.

In one embodiment of the method, the modular synthetic hydrogel comprises polyalginate and hyaluronan.

In one embodiment, the combined concentration of PEG and at least one of heparain, hyaluronan, and collagen in the modular synthetic hydrogel is from about 0.05% (w/w) to about 98% (w/w) based on the total weight of the modular synthetic hydrogel.

In some embodiments the modular synthetic hydrogel comprises PEG and hyaluronan. In other embodiments the modular synthetic hydrogel comprises PEG and collagen

In one embodiment of the method the modular synthetic hydrogel is a StarPEG hydrogel. In other embodiments the modular synthetic hydrogel is a linear PEG hydrogel.

In one embodiment, the present specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the cell population secretes an extracellular matrix into the modular synthetic hydrogel, and wherein, at the time of administration, the concentration of the extracellular matrix is about 0.05% (w/w) to about 98% (w/w) based on the total weight of the modular synthetic hydrogel.

In another embodiment, the synthetic tissue further comprises one or more exogenous growth factors, antibodies or cell penetrating peptide (CPP) fusion proteins.

In one embodiment, the one or more exogenous growth factors are selected from the group consisting of: BDNF, VEGF, IGF1, bFGF/FGF2, Ang1, Ang 2, BMP 2, BMP 3a, BMP 3b, BMP 4, BMP 5, BMP 6, BMP 7 (OP-1), CTNF, EGF, EPO, aFGF/FGF1, bFGF/FGF2, G-CSF, GDF10, GDF15, GDNF, GH, GM-CSF, HB-EGF, LIF, NGF, NT-3, NT 4/5, Osteopontin, PDGFaa, PDGFbb, PDGFab, P1GF, SCF, SDF1/CXCL12, and TGFβ.

In one embodiment, the CPP fusion proteins include one or more CPP-transcription factor-CPP fusion proteins.

In some embodiments the synthetic tissue further comprises one or more exogenous exosomes.

In some embodiments the methods described herein further include administering to the subject one or more exogenous growth factors, antibodies or cell penetrating peptide (CPP) fusion proteins.

In one embodiment, the specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the synthetic tissue is provided as a construct having a predefined shape prior to the administration.

In one embodiment, the predefined shape of the construct is customised based on the shape of the site where the tissue construct is to be implanted.

In one embodiment, the synthetic tissue comprises a homogeneous modular synthetic hydrogel having the same density/viscosity throughout.

In one embodiment, the synthetic tissue comprises a heterogenous modular synthetic hydrogel having regions or layers or gradients of different density/viscosity.

In one embodiment, the modular synthetic hydrogel comprises a cell population within synthetic hydrogel microparticles beads or other shapes having a volume of about 0.2 μl.

In one embodiment, the synthetic tissue construct is implanted into or proximal to the brain, spinal cord, optic nerve, or a peripheral nerve of the subject.

In one embodiment, the specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the synthetic tissue is provided as a construct having a predefined shape prior to the administration and wherein the construct is obtained by 3D tissue printing.

Accordingly, in one embodiment, the method further comprises forming the construct into a predefined shape. In one embodiment, the method further comprises forming the construct into a predefined shape by 3D printing. In one embodiment, modular synthetic hydrogels of different physical characteristics such as for example, density, viscosity, are manufactured in pre-determined shapes comprising heterogeneous or homogenous modular synthetic hydrogels. Heterogeneous modular synthetic hydrogels and microparticles comprising same may be manufactured in the form of inter-unit layers, folds, gradients, pockets, regions and the like, in the same micro-tissue unit.

In another embodiment, the specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the synthetic tissue is provided in the form of microparticles.

In one embodiment, the microparticles have a diameter of about 0.1 μm to about 2000 μm.

In one embodiment of the method, the microparticles have a diameter of about 0.2 μm to about 1000 μm, or 2 μm to about 700 μm, or 1.5 μm to about 900 μm.

In one embodiment of the method, microparticles have a diameter of about 50 μm to about 500 μm.

In one embodiment of the method, the microparticles comprise at least first and second populations of microparticles that differ from each other in at least one of the following characteristics: cell types, proportions of cell types, spatial distribution of cell types, hydrogel subunit materials, linked peptides or polypeptides, exogenous growth factors.

In one embodiment of this aspect of the method, the first and second populations of microparticles are administered at different time points or different sites in the subject.

In one embodiment, the synthetic tissue is administered as an aqueous suspension having a viscosity greater than 100 Pa.

In one embodiment, the synthetic tissue was generated by one or more liquid handling robots.

In one embodiment the method further comprises generating the synthetic tissue using one or more liquid handling robots.

In one embodiment, the modular synthetic hydrogel in the synthetic tissue is generated by spray polymerisation.

In one embodiment, the synthetic tissue is cultured for about two weeks to about three months.

Accordingly, in one embodiment, the specification provides a method comprising administering to the subject a therapeutically effective amount of a synthetic tissue comprising a cell population comprising (a) one or more nervous system cell types, or (b) multipotent cells; wherein the cell population is embedded within a modular synthetic hydrogel that is biocompatible, wherein the synthetic tissue is cultured for about two weeks to about three months.

In one embodiment of the method, the synthetic tissue is injected into or proximal to the brain, spinal cord, optic nerve, or a peripheral nerve of the subject.

In one embodiment of the method, the subject is a human subject.

In one embodiment of the method, the synthetic tissue is provided for the administration within a secondary modular synthetic hydrogel layer that is biocompatible.

In accordance with this aspect, the method further comprises generating the secondary modular synthetic hydrogel layer in the presence of the synthetic tissue.

In some embodiments, at the end of a culture period of a synthetic tissue, the modular synthetic hydrogel in which the cell population was embedded has been substantially degraded prior to the administration of the synthetic tissue to a subject.

The present specification provides a synthetic tissue for use or when used in treating a neurological disorder or repairing nervous system tissue, wherein the synthetic tissue comprises a cell population comprising (a) one or more nervous system cell types, or (b) multipotent cells, and wherein the cell population is embedded within a modular synthetic hydrogel that is biocompatible.

In a related aspect provided herein is a synthetic tissue for use in treating a neurological disorder, wherein the synthetic tissue was obtained by a process comprising the steps of:

(i) embedding a cell population comprising (a) one or more nervous system cell types, or (b) multipotent cells in a modular synthetic hydrogel; and

(ii) culturing the embedded cell population for a period.

Similarly, the present specification provides for the use of a synthetic tissue in the manufacture of a medicament for treating a neurological disorder or repairing nervous system tissue, wherein the synthetic tissue comprises a cell population comprising (a) one or more nervous system cell types, or (b) multipotent cells, and wherein the cell population is embedded within a modular synthetic hydrogel that is biocompatible.

In a further aspect provided herein is the use of a synthetic tissue in the manufacture of a medicament for treating a neurological disorder, wherein the synthetic tissue was obtained by a process comprising the steps of :

(i) embedding a cell population comprising (a) one or more nervous system cell types, or (b) multipotent cells in a modular synthetic hydrogel; and

ii) culturing the embedded cell population for a period.

Any embodiment herein shall be taken to apply mutatis mutandis to any other embodiment unless specifically stated otherwise. For instance, as the skilled person would understand the use of peptides with protease cleavage sites in PEG-based modular synthetic hydrogels, applies equally in other synthetic polymer (e.g., polyvinyl alcohol)-based modular synthetic hydrogels above for use in the methods of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—Schematic illustration of steps in an exemplary, non-limiting, embodiment of synthetic tissue generation and administration. (1) A population of one or more nervous system cell types (e.g., dopaminergic neurons) in desired proportions is resuspended in a physiological buffer; (2) The cell population is mixed with a functionalized (reactive) biopolymer such as a glycosaminoglycan (e.g., Heparin) containing multiple maleimide groups (HEP-maleimide); (3) a synthetic polymer linked to at least one biocleavable peptide comprising a reactive thiol group (e.g., a cysteine residue) such as StarPEG-metalloprotease cleavage site peptide (MMP) is combined with the HEP-maleimide, cell mixture. The thiol groups in the Star-PEG-linked peptide(s) rapidly react with the maleimdide group(s) on the HEP-maleimide via Michaels addition under physological conditions resulting in hydrogel assembly and embedding of the cells within the hydrogel matrix. See also FIG. 2. Optionally, the Star-PEG or HEP-maleimide are functionalized with peptides that include a cell binding sequence (e.g., an RGD motif) to promote cell adhesion and motility within the gel matrix. Optionally, the Cell HEP-maleimide and StarPEG-peptide mix is automated by use of a liquid handling robot to rapidly generate (4) synthetic brain micro-tissue beads (AKA “microparticles”) of a desired size (e.g., 250 μm) also referred to as “SBMs”; (5) SBMs are plated into cell culture vessels in a desired format and in suitable culture medium; (6) Extended culture of SBMs promotes cell differentiation, functional maturation (e.g., secretion of cognate neurotransmitters and synapse formation). In addition, over an extended culture period cell-mediated metalloprotease cleavage of target linked peptides and secretion of extracellular matrix (ECM) proteins promotes generation of a tissue-mimetic environment within the SBMs; (7) after a desired culture/maturation period, SBMs are pooled in preparation for administration within a suitable physiological buffer. Optionally, the pooled SBMs are embedded in a secondary hydrogel layer of lower viscosity, which reduces dispersal of the SBMs once administered and promotes integration of the SBMs with surrounding host nervous system tissue; (8) The synthetic tissue, e.g., as SBMs is injected intracranially at a site of injury or cell deficiency into a subject suffering from a neurological condition (e.g., Parkinson's disease) through a syringe or catheter in a desired does depending on the desired number of cells and synthetic tissue volume to be administered.

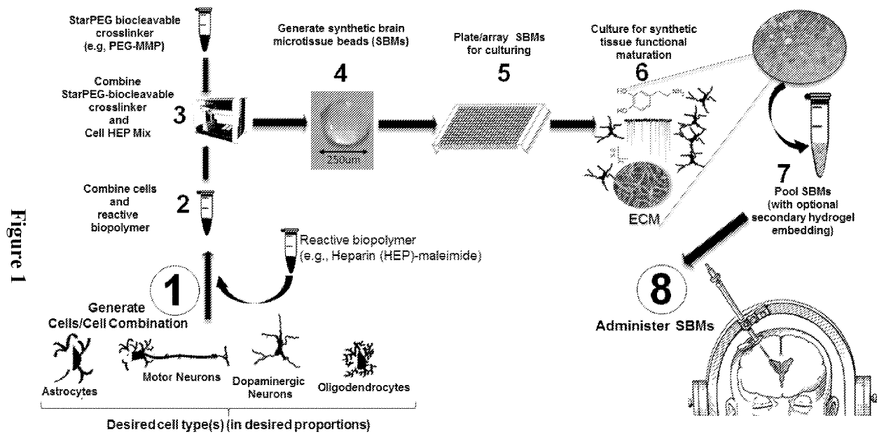

FIG. 2—Schematic illustration of: (A) an exemplary, non-limiting list of PEG architectures. “R” at the end of each PEG arm denotes functionalisation with a peptide and/or reactive (bio)molecule; (B) exemplary embodiment of synthetic tissue generation in which cells (optionally in combination with growth factors or other peptides) are embedded in a hydrogel formed by crosslinking of bifunctional PEG a maleimide-functionalised glycosaminoglycan such as hyaluronic acid or collage.

FIG. 3—Bright-field images of functional synthetic human brain tissue from a synthetic brain micro-tissue beads (AKA “microparticles”) of a desired size (e.g., 250 μm) also referred to as “SBMs”; plated into cell culture vessel in suitable culture medium; manufactured using the methods described herein; (A) synthetic human brain tissue composed of human neurons and manufactured using the described methods; (B) magnification of synthetic human brain tissue encompassed by rectangular region of interest denoted in (A). The arrow designated “1”, points to an example of human neuron, the cellular building block of the tissue that appears in this figure. Arrow “ii”, indicates a layer of synthetic human brain tissue structure, reminiscent of the actual human brain tissue layers. Curved line designated “iii”, highlights a synthetic human brain tissue “fold,” reminiscent of naturally occurring folds observed in native human brain tissue. (C), At higher magnification individual neurons and processes (designated by set of four arrows) are observed, which form a network of interconnected human neurons within the synthetic human brain tissue during culture as described.

### DETAILED DESCRIPTION OF PARTICULAR EMBODIMENTS

### General Techniques and Definitions

Unless specifically defined otherwise, all technical and scientific terms used herein shall be taken to have the same meaning as commonly understood by one of ordinary skill in the art (e.g., in cell culture, cell biology, molecular genetics, neuroscience, immunology, pharmacology, protein chemistry, and biochemistry).

Unless otherwise indicated, the cell culture and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the term about, unless stated to the contrary, refers to ±10%, more preferably ±5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “hydrogel subunit material,” as used herein refers to an inert synthetic polymer (e.g., PEG and/or PVA) that is biocompatible and can be derivatised and/or cross-linked to form a hydrogel.

The term “modular synthetic hydrogel,” as used herein, refers to hydrogel containing: (a) a polymeric scaffold that has been derivatised to add multiple functional groups to form covalent or non-covalent bonding; and (b) linking peptides or polypeptides (e.g., glycosaminoglycans such as heparin) that have been derivatised with multiple functional groups that can form covalent or non-covalent bonds with other peptides (e.g., cell adhesion peptides) and with the polymeric scaffold in order to crosslink it.

The term “synthetic tissue,” or “synthetic brain micro-tissue (SBM)” as used herein, refers to a 3D construct comprising a population of cells embedded in a modular synthetic hydrogel.

The term “peptide,” as used herein, refers to a polymer of amino acids ranging from two to about fifty amino acids (e.g., 4, 6, 8, 10, 12, 15, 20, 25, 30, 35, 40, or 45 amino acids in length). The term peptide encompasses both unmodified peptides, phosphorylated peptides (e.g., phosphopeptides), and otherwise chemically derivatized peptides, but not peptidomimetics.

The term “polypeptide,” or “protein” as used herein, refer to a polymer of amino acids generally greater than about 50 amino acids in length and typically having table characteristic secondary and tertiary structures.

As the skilled person would understand, a synthetic tissue, as described herein, will be administered in a therapeutically effective amount. The terms “effective amount” or “therapeutically effective amount,” as used herein, refer to a sufficient amount of a synthetic tissue being administered which will relieve to some extent one or more of the symptoms of the disease or condition being treated. The result can be reduction and/or alleviation of the signs, symptoms, or causes of a neurological disorder, or any other desired alteration of a biological system. For example, an “effective amount” for therapeutic uses is the amount of the synthetic tissue required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a synthetic tissue is an amount effective to achieve a desired therapeutic improvement without undue adverse side effects. It is understood that “an effect amount” or “a therapeutically effective amount” can vary from subject to subject, due to variation in a number of factors including, but not limited to, the age, and general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “small molecule therapeutic agent” as used herein, refers to a pharmacological agent having a molecular weight below 2000 daltons and approved for therapeutic use in humans.

The terms “treating” or “treatment,” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

### Methods of Treatment

Some methods described herein include repair of nervous system tissue in a subject in need by administering a therapeutically effective amount of a biocompatible, synthetic tissue comprising a cell population that includes either (a) one or more nervous system cell types (e.g., neurons, neural progenitors, astrocytes or (b) multipotent cells, wherein the cell population is embedded within a modular synthetic hydrogel. The methods described herein also include treating a neurological disorder by administration of the above-mentioned synthetic tissue. Nervous system repair is facilitated by the use of modular, synthetic hydrogels to promote functional maturation and connectivity of cells within the hydrogel matrix, ex vivo. In particular, as described herein, the hydrogel matrix is crosslinked, in part, with cell-cleavable peptides such that in culture, the matrix is progressively reconfigured by maturing cell populations, and extracellular matrix is deposited to provide a tissue-mimetic environment.

In some embodiments methods for repairing nervous system tissue or for treating a neurological disorder in a subject include administering to the subject a therapeutically effective amount of a synthetic tissue obtained by a process comprising the steps of:

(i) embedding a cell population comprising (a) one or more nervous system cell types, or (b) multipotent cells in a modular synthetic hydrogel; and

(ii) culturing the embedded cell population for a period.

In some embodiments, following the culture period, the modular synthetic hydrogel in which the cell population was embedded has been substantially degraded prior to the administration. In some embodiments, about 10% (by mass) to substantially all of the modular synthetic hydrogel has been degraded prior to administration of a synthetic tissue to a subject, e.g., 15%, 20%, 25%, 30%, 40%, 50%, 60%, 70%, 80%, 90%, or another percent from about 10% of the synthetic hydrogel (by mass) to substantially all of the modular synthetic hydrogel has been degraded prior to administration of the synthetic tissue.

Without being bound by theory, it is believed that use of such synthetic tissues greatly facilitates, relative to previous cell therapy or explant approaches, the localization and functional integration of the component cells into host nervous system tissue. Further, in some cases, this integration may be facilitated by degradation of the modular synthetic hydrogel by an embedded cell population paralleled by secretion of extracellular matrix proteins by the embedded cell population.

Nervous system repair can be necessitated by, e.g., trauma (e.g., a head injury), surgical wounds (e.g., brain tumour removal), and implantation of a medical device (e.g., a vagal nerve stimulator).

Examples of neurological orders suitable for treatment by the methods of the invention include, but are not limited to, Alzheimer's Disease, vascular dementia, Parkinson's Disease, Huntington's Disease, stroke, ischemic stroke, haemorrhagic stroke, optic nerve disease, spinal cord injury, peripheral nerve injury, demyelinating disease, traumatic brain injury, ataxia, or Frontotemporal dementia. In some preferred embodiments a subject to be treated is a human subject suffering from Parkinson's disease.

In some embodiments the subject to be treated is suffering from a surgical wound occurring in nervous system tissue (e.g., brain or spinal cord tissue) or is suffering from other damage associated with complication of surgery.

Symptoms, diagnostic tests, and prognostic tests for each of the above-mentioned conditions are known in the art. See, e.g., Harrison's Principles of Internal Medicine®,” 19th ed., Vols 1 & 2, 2015, The McGraw-Hill Companies, Inc.

As used herein, the term “subject” can be any animal. Exemplary subjects include but are not limited to human, non-human primates (e.g. macaques, tree shrews, chimpanzees). In preferred embodiments, the subject is a human.

A number of animal models are useful for establishing a range of therapeutically effective doses of the synthetic tissue described herein for treating any of the foregoing neurological disorders. For example, a number of animal models of Parkinson's disease have been established (Blesa et al., 2014), for stroke (McCabe et al, 2018), for Alzheimer's disease (Gotz et al, 2018), and for traumatic brain injury (Hadjigeorgiou et al., 2017).

In some embodiments a therapeutically effective dose of synthetic tissue comprises a total number of cells (e.g., neurons and glial cells) of about 1×10⁵ cells to about 1×10⁹ cells, e.g., 3×10⁵, 4×10⁵, 5×10⁵, 7×10⁵, 1×10⁶, 3×10⁶, 5×10⁶, 8×10⁶, 1×10⁷, 2×10⁷, 3×10⁷, 4×10⁷, 5×10⁷, 7×10⁷, 1×10⁸, 2×10⁸, 3×10⁸, 5×10⁸, 7×10⁸, or another total number of cells from about 1×10⁵ cells to about 1×10⁹ cells.

In some embodiments the therapeutically effective dose of synthetic tissue to be administered comprises about 5×10⁵ cells to about 1×10⁷ cells, e.g., 3×10⁵, 4×10⁵, 5×10⁵, 7×10⁵, 1×10⁶, 3×10⁶, 5×10⁶, 8×10⁶, or another number of cells from about 5×10⁵ cells to about 1×10⁷ cells.

In some embodiments the total administration volume within which the synthetic tissue is provided ranges from about 0.2 μl to about 1000 μl, e.g., 0.2 μl, 5 μl, 7 μl, 10 μl, 12 μl, 20 μl, 25 μl, 35 μl, 50 μl, 75 μl, 100 μl, 200 μl, 300 μl, 400 μl, 500 μl, 700 μl, 800 μl, or another total administration volume from about 0.2 μl to about 1000 μl.

In some embodiments the synthetic tissue is administered in multiple doses administered simultaneously (or over a short period of time) or at appropriate intervals, for example as two, three, four or more doses over a period of about one day to about seven (7) years.

The foregoing ranges are merely suggestive, as the number of variables in regard to an individual treatment regime is large, and considerable excursions from these recommended values are not uncommon. Such dosages may be altered depending on a number of variables, not limited to the neurological condition to be treated, the number of sites of administration within nervous system tissue, the type of cells being administered, and the characteristics of the modular synthetic hydrogel utilised in the synthetic tissue as described herein.

In preferred embodiments administration of a synthetic tissue is through a local route of administration into a central or peripheral nervous system site. In some embodiments the synthetic tissue construct is implanted into or proximal to the brain, spinal cord, optic nerve, or a peripheral nerve of the subject.

In some embodiments a subject to be treated, in addition to being administered a synthetic tissue as described herein, is also administered one or more exogenous growth factors, antibodies or cell penetrating (CPP) fusion proteins as described herein.

### Synthetic Tissue for use in Treatment Methods

Synthetic tissues for use in the methods described herein comprise a population of one or more nervous system cell types, or multi-potential cells, cultured in 3D within a modular synthetic hydrogel.

In some embodiments, the modular synthetic hydrogel to be used comprises nervous system cell types. Suitable nervous system cell types for culture within such modular synthetic hydrogels include, but are not limited to neurons, neural progenitor cells, glial cells, and any combination thereof. In some embodiments the synthetic tissue comprises neurons.

Depending on the particular condition to be treated, suitable types of neurons include, but are not limited to, monoaminergic neurons, catecholaminergic neurons, glutamatergic excitatory neurons, GABAergic inhibitory neurons, motor neurons, cholinergic neurons, or any combination thereof. In some preferred embodiments, particularly where a subject to be treated is suffering from Parkinson's disease, the cell population in the synthetic tissue comprises A9-subtype ventral midbrain dopaminergic neurons. In some embodiments the cell population comprises both excitatory neurons (e.g., glutamatergic neurons) and inhibitory neurons (e.g., GABAergic neurons). While not wishing to be bound by theory, the presence of both excitatory and inhibitory neurons in the synthetic tissues described herein is believed to promote the formation of functional circuits that exhibit more physiological levels and patterns of activity relative to circuits comprising solely excitatory or inhibitory neurons. In some embodiments, where both excitatory and inhibitory neurons are to be included in the cell population, they are provided within the synthetic tissue in a predetermined ratio. In some embodiments the ratio of inhibitory neurons to excitatory neurons is about 1:20 to about 1:1, e.g., 1:12, 1:8, 1:5, 1:3, 1:2, or another ratio of inhibitory neurons to excitatory neurons from about 1:20 to about 1:1. In some embodiments the ratio of inhibitory neurons to excitatory neurons in the synthetic tissue to be administered is about 1:5.

In some embodiments, the modular synthetic hydrogel is seeded with a cell population that includes or consists of multipotent neural progenitor cells (NPCs). In some embodiments, the NPCs are cultured in the modular synthetic hydrogel, prior to administration, to promote differentiation into neurons, astrocytes, or combinations thereof. In other embodiments, NPCs are allowed to proliferate within the modular synthetic hydrogel and are the prevalent cell type present in the synthetic tissue prior to administration with a view to allowing differentiation of the NPCs to occur in vivo after administration of the synthetic tissue.

Cells for inclusion in the synthetic tissue for administration as described herein can be obtained as primary cells from a variety of sources including, e.g., fetal tissues or adult tissues. Alternatively, such cells can be obtained indirectly by differentiation of pluripotent or multipotent cell types such as induced pluripotent stem cells (iPSCs), embryonic stem cells. In other embodiments, differentiated cells can also be obtained by direct lineage reprogramming of somatic cells.

In other embodiments the synthetic tissue comprises glial cells. Suitable types of glial cells include, but are not limited to, astrocytes, myelinating glial cells (e.g., oligodendrocytes and Schwaan cells), or microglia. In some embodiments the population of cells in the synthetic tissue to be administered comprises myelinating glial cells. In some embodiments the synthetic tissue comprising myelinating glial cells is administered to a subject suffering from a spinal cord injury or traumatic brain injury.

In some embodiments the population of cells in the synthetic tissue to be administered comprises both neurons and glial cells. In some embodiments the synthetic tissue comprises neurons and glial cells in a predetermined ratio. Suitable ratios of glial cells to neurons range from about 7:1 to about 1:5, e.g., 5:1, 4:1, 3:1, 2:1, 1:1, 1:2, 1:3 or another ratio of glial cells to neurons from about 7:1 to about 1:5.

In some embodiments the population of cells further comprises endothelial cells. While not wishing to be bound by theory, it is believed that inclusion of endothelial cells may promote angiogenesis in synthetic tissue and thereby promote its survival when implanted in vivo.

Cell separation of cells having required lineage/cell type markers can be accomplished by, e.g., flow cytometry, fluorescence-activated cell sorting (FACS), or, preferably, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. Magnetic cell separation can be performed and automated using, e.g., an AUTOMACS™. Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies.

In some preferred embodiments, where the population of cells in the synthetic tissue to be administered includes neurons, the synthetic tissue is cultured under conditions and for a sufficient period of time to exhibit one or more functional characteristics associated with neuronal maturation. Such functional characteristics include, but are not limited to, secretion of a cognate neurotransmitter, secretion of a growth factor, expression of a mature neuronal protein marker, surface expression or subcellular localisation of a neurotransmitter receptor, intrinsic electrical activity, and synaptic connectivity.

For example mature A9 mature midbrain dopaminergic neurons can be assayed for secretion of dopamine, expression of Tyrosine hydroxylase, expression of Dopamine transporter (DAT), expression of the transcription factor FOXA2, expression of the G-protein-regulated potassium channel GIRK2, expression of the transcription factor Nurr1, and expression of the transcription factor LMX1B.

Glutamatergic neurons can be characterised by assaying expression of the glutamate transporter (vGlut) transporters, NMDA receptors, AMPA receptors, and glutaminase. GABAergic neurons can be characterised by assaying expression of GABA transporters, GABA receptors, Glutamate decarboxylase (e.g., GAD65 or GAD67).

Motor neuron identity can be confirmed by assaying expression of the transcription factor HB9 and/or choline acetyltransferase (ChAT).

Electrophysiological maturation is generally characterised by a progressively increasing membrane potential over the culture period, a decrease in input resistance reflecting increasing complexity in cell shape and an associated dendritic arbor, the development of evoked and spontaneous action potentials, and spontaneous synaptic activity, e.g., the presence of AMPA and/or NMDA receptor-mediated excitatory postsynaptic currents, and/or inhibitory postsynaptic currents. In some embodiments the synthetic tissue to be administered is characterised by the presence of both excitatory and inhibitory neurotransmission.

In some preferred embodiments, where particular types of neurons (e.g., dopaminergic neurons) are to be administered, the ability of the neurons to undertake synaptic release of a cognate neurotransmitter can be assessed prior to ensure that the cells are suitable for administration. For example, the release of dopamine from cultured dopaminergic neurons or acetylcholine from cholinergic neurons can be assessed by any of a number of standard techniques, e.g., ELISA. In some embodiments the synthetic tissue comprises neurons that secrete a cognate neurotransmitter. In some embodiments the neurons administered in the synthetic tissue secrete dopamine, acetylcholine, or serotonin.

In some embodiments, the synthetic tissue to be administered is initially seeded with a cell population that includes or consists of multipotent neural progenitor cells (NPCs). In some embodiments, the NPCs are cultured in the modular synthetic hydrogel, prior to administration, to promote differentiation into neurons, astrocytes, or combinations thereof. In other embodiments, NPCs are allowed to proliferate within the modular synthetic hydrogel and are the prevalent cell type present in the synthetic tissue prior to administration with a view of allowing differentiation of the NPCs to occur in vivo after administration.

In other embodiments the population of cells to be included in the synthetic tissue comprises multipotent cells. Suitable multipotential cells include, but are not limited to, mesenchymal stem cells.

In some embodiments, the synthetic tissue comprising a modular synthetic hydrogel and a cell population as described above is administered immediately after manufacture or without a culture period to allow for cell maturation/proliferation/differentiation.

In some embodiments after the initial formation of a synthetic tissue comprising a modular synthetic hydrogel and a cell population as described above, the resulting synthetic tissue is cultured for a period of time to allow differentiation, maturation, and/or proliferation of cells in the embedded population. In some embodiments the culture period is about 7 days to about 120 days, e.g., 14 days, 21 days, 28 days, 40 days, 50 days, 60 days, 70 days, 80 days, or another cell culture period from about 7 days to about 90 days. In some preferred embodiments the synthetic tissue is cultured from about 14 days to about 60 days. The skilled person will appreciate that a suitable culture period prior to administration of the synthetic tissue will be determined based on a number factors including, but not limited to, the nervous system cells present in the initial cell population, the level of neuronal maturation desired, and the necessity of allowing the cells to proliferate within the synthetic tissue. For example, where the initial (seeding) cell population contains predominantly NPCs that are to be differentiated ex vivo, the required culture period will be longer than when seeding with pre-differentiated cells, as NPCs must typically undergo an extended culture period to obtain differentiated neurons, and even longer for astrocytes. A number of protocols, especially for human cells, are known for neuronal differentiation and patterning of NPCs. See, e.g., Studer et al. (2012), Shi et al. (2012); and for glial differentiation, e.g., Santos et al. (2017).

In some embodiments the population of cells in the synthetic tissue includes genetically modified cells. In some embodiments such genetically modified cells express one or more exogenous proteins including, but not limited to, one or more of light-sensitive ion channels, chemogenetically engineered proteins, reporter proteins, optogenetic probe proteins, growth factors, transcription factors, antibodies, and cytokines including pro-inflammatory and anti-inflammatory cytokines. In other embodiments genetically modified cells express exogenous RNAs including, e.g., exosomal mRNAs and non-coding RNA(e.g., miRNAs)

In some embodiments the population of cells in the synthetic tissue to be administered includes cells that are allogeneic with respect to the subject to be treated. In other embodiments the population of cells in the synthetic tissue includes autogeneic cells. In yet other embodiments the population of cells includes both autogeneic and allogeneic cells.

As will be appreciated by the person of ordinary skill in the art, populations of cells in the nervous system are frequently found to be distributed in specific spatial patterns and/or circuits. For example, mammalian neocortex is divided into six layers having distinctive proportions of cell type and connectivity that underly its function. Accordingly, in some embodiments the cell population embedded within a modular synthetic hydrogel as described herein is distributed non-uniformly within a volume of the modular synthetic hydrogel. In some embodiments, where the cell population is to be distributed non-uniformly, the cell population has a predetermined spatial distribution. Examples of such, such a predetermined spatial distribution include, but are not limited to, layers, clusters, or concentric spheres, of cells with different cell types or different proportions of cell types and/or connectivity. In some embodiments the synthetic tissue is provided as a construct having a predefined shape prior to administration by implantation. In some preferred embodiments, where the method utilises a synthetic tissue having a predefined shape, the predefined shape is customised based on the shape of a site within the host tissue where the synthetic tissue construct is to be implanted. In some embodiments the predefined shape of the synthetic tissue construct was obtained by 3D tissue printing. In some embodiments the methods described herein include the step of 3D tissue printing of the synthetic tissue construct.

In other embodiments the synthetic tissue is provided in the form of synthetic tissue microparticles, also referred to herein as “synthetic brain microtissue beads” (SBMs). In some embodiments such tissue microparticles have a diameter of about 1 μm to about 2000 μm, e.g., 1.5 μm 10 μm, 20 μm, 30 μm, 50 μm, 100 μm, 200 μm, 300 μm, 400 μm, 500 μm, 600 μm, 700 μm, 800 μm, 1000 μm, 1200 μm, 1500 μm, 1700 μm, or another diameter from about 1 μm to about 2000 μm.

In some embodiments, where the synthetic tissue to be administered is provided in the form of tissue microparticles, such tissue microparticles include at least two different populations of microparticles that differ from each other in at least one of the following characteristics: cell types, proportions of cell types, spatial distribution of cell types, hydrogel subunit materials, linked peptides or polypeptides, exogenous growth factors. In some embodiments such multiple populations of tissue microparticles having diverse characteristics are administered together. In other embodiments multiple populations of tissue microparticles are administered at different time points from one another, e.g., with a time interval from about 1 day to about 2 months, e.g., 3 days, 7 days, 14 days, 21 days, 1 month, 40 days, 50 days, or another time interval from about 1 day to about 2 months.

Depending on the particular site of administration, the desired localisation, and a neurological condition to be treated, in some embodiments the synthetic tissue microparticles are administered as an aqueous suspension has a viscosity greater than 100 Pa, e.g., from about 120 Pa to about 700 Pa, e.g., 150 Pa, 200 Pa, 300 Pa, 400 Pa, 500 Pa, 550 Pa, 600 Pa, or another viscosity level from about 120 Pa to about 700 Pa.

In some embodiments, the synthetic tissue is provided for administration within a secondary modular synthetic hydrogel layer that is biocompatible. In some embodiments the methods described herein also include the step of forming the secondary modular synthetic hydrogel around the synthetic tissue to be administered. Typically, the secondary modular synthetic hydrogel layer will be of lower viscosity than the synthetic tissue it encompasses. While not wishing to be bound by theory, it is believed that the secondary modular synthetic hydrogel layer can reduce dispersal of cells away from the tissue administration site and promote integration of the administered synthetic tissue within the surrounding host nervous system tissue.

### Modular Synthetic Hydrogels

The methods described herein exploit the advantages of modular synthetic hydrogels, which, as referred to herein, are extracellular matrix (ECM)-inspired polymer hydrogels that combine an inert, synthetic hydrogel polymer, e.g., poly(ethyleneglycol) (PEG) with cell adhesive and/or cell degradable peptide crosslinkers, and multi-functionalised linking polypeptides (e.g., maleimide-derivatised glycosaminoglycans) to form a modular synthetic hydrogel. Such hydrogels afford precise and independent control over the reactivity and specificity of multiple functional components and the polymer network properties (e.g., stiffness) as described in, e.g., Tsurkan et al.(2013). An important advantage of such hydrogels is that while initially providing a suitable adhesive substrate for the cells used in the methods described herein, they allow progressive cleavage of at least a portion of linked peptides within the hydrogel scaffold. Thus, over time cell-secreted ECM deposited into the modular hydrogel enhances the tissue-mimetic 3D network properties of the modular synthetic hydrogel to promote differentiation and functional maturation of an embedded cell population.

### Hydrogel Subunit Materials

Suitable hydrogel subunit materials for the generation of an inert synthetic hydrogel polymer include, but are not limited to, one or more of polyethylene glycol (PEG), hyaluronan, gelatin, thiol-modified hyaluronan, acrylated hyaluronic acid thiol-modified chondroitin sulfate, thiol-modified gelatin, acrylic copolymers, polyvinylidene fluoride, chitosan, polyurethane isocyanates, polyalginate, cellulose acetate, polysulfone, polyvinyl alcohols, and polyacrylonitrile. In some embodiments the hydrogel subunit materials in the modular synthetic hydrogel include a multi-arm (AKA “star”) polymer. In other embodiments the hydrogel subunit materials in the modular synthetic hydrogel include any of a linear, bifunctional, Y-shaped, or fork-shaped polymer. In some embodiments the hydrogel subunit materials in the modular synthetic hydrogel include PEG. In some embodiments the modular synthetic hydrogel includes a starPEG. In other embodiments, the modular synthetic hydrogel includes a linear PEG. In other embodiments the hydrogel subunit materials in the modular synthetic hydrogel include hyaluronan.

Generally the hydrogel subunit materials to be used in the generation of modular synthetic hydrogels used in the methods described herein are functionalised with maleimide groups or other functional groups to allow conjugation to one or more first linking peptides via terminal thiol groups through a Michael addition reaction under mild conditions, as described in Tsurkan et al. (2013). The resulting hydrogel polymer-peptide conjugates can the be further reacted through thiol-containing cysteine groups in the first linked peptides with a maleimide-functionalised second linking peptide or polypeptide such as maleimide-functionalised glycosaminoglycan (GAG) (e.g., maleimide-heparin).

### Linking Peptides and Polypeptides

In some embodiments the modular synthetic hydrogels used in the methods described herein include one or more peptides or polypeptides (“linking peptides”) linked to the one or more hydrogel sub-unity materials (e.g., PEG). Such peptides provide a crosslinking function by their ability to react through thiol-containing cysteine groups with maleimide, or similar functional groups to form covalent links on hydrogel scaffold materials or peptides that have been derivatised with maleimide as known in the art. In addition such peptides optionally include other functionalities such as cell adhesion and protease cleavage sites.

Accordingly, in some embodiments the modular synthetic hydrogel includes one or more peptides linked to the one or more constituent hydrogel subunit materials. In some preferred embodiments the one or more linking peptides comprise at least a first and a second peptide or polypeptide. In some embodiments at least one of the linking peptides or polypeptides is enzymatically cleavable. In some embodiments at least one of the linking peptides or polypeptides is cleavable by a metalloprotease. In some embodiments the first linking peptide comprises the following metalloprotease cleavage recognition sequence:

(SEQ ID NO: 1)
GPQGIWGQGGCG

Other suitable enzymatic cleavage recognition sequences are known in the art. While not wishing to be bound by theory, it is believed that the inclusion of sequences allowing for cell-mediated enzymatic cleavage of crosslinking peptides promotes formation of a more in vivo like extracellular matrix by resident cells, and promotes their differentiation or maturation.

In some embodiments the at least one of the linking peptides or polypeptides includes a cell binding sequence. Suitable cell binding sequences include laminin-derived cell binding sequences and RGD motif peptides. In some embodiments the cell binding sequence includes one of the following amino acid sequences:

(SEQ ID NO: 2)
SIKVAVGWCG
(SEQ ID NO: 3)
YIGSRGCG

In some embodiments at least one of the linking peptides or polypeptides includes a convertible functional group such as a malemide group or thiol group.

In some embodiments at least one of the linking peptides is a glycosaminoglycan (GAG). In some preferred embodiments the GAG is heparin or a heparin derivative (e.g., thiol-modified heparin). Functionalisation of linking peptides and polypeptides with reactive groups such as maleimide or thiol, including, region-selective functionalisation, is known in the art as described in, e.g., Tsurkan et al. (2013).

In some embodiments the modular synthetic hydrogel includes PEG and heparin. In other embodiments the modular synthetic hydrogel includes PEG and collagen. In other embodiments the modular synthetic hydrogel includes PEG and hyaluronan. In yet other embodiments the modular synthetic hydrogel includes PEG and a combination of heparin, collagen, and hyaluronan

In other embodiments the modular synthetic hydrogel includes polyalginate and at least one of hyaluronan, heparin, and collagen.

In some embodiments the combined concentration of PEG and heparin, collagen, and/or hyaluroan in the modular synthetic hydrogel is from about 0.05% (w/w) to about 98% (w/w) based on the total weight of the modular synthetic hydrogel, e.g., a concentration of 0.1%, 0.2%, 0.4%, 0.5%, 1%, 10%, 20%, 30%, 40%, 50%, 60%, 75%, 80%, 90%, 95% or another concentration (w/w) from about 0.05% to about 98%. In some preferred embodiments, where the modular synthetic hydrogel comprises starPEG (or peptide-linked heparin) and heparin (or peptide-linked heparin), the molar ratio of starPEG to heparin is about 0.5 to about 1.5, e.g., 0.6, 0.7, 0.75, 0.8, 0.9, 1.0, 1.2, 1.2, 1.3 or another molar ratio of starPEG to heparin from about 0.5 5 to about 1.5. In one preferred embodiment the molar ratio of starPEG to heparin is about 0.75 to 1.0.

The skilled person will appreciate that cells embedded and cultured within the modular synthetic hydrogels, particularly ones functionalised with enzymatically cleavable linking peptides, will over time secrete an extracellular matrix that will contribute an increasing proportion of the total synthetic hydrogel mass over a given culture period. Accordingly, in some embodiments the concentration of the extracellular matrix ranges from about 0.05% (w/w) to about 98% (w/w) based on the total weight of the modular synthetic hydrogel, e.g., 0.1%, 0.2%, 0.4%, 0.5%, 1%, 10%, 20%, 30%, 40%, 50%, 60%, 75%, 80%, 90%, 95% or another concentration (w/w) from about 0.05% to about 98%.

### Optional Agents for Release from Modular Synthetic Hydrogels

In some embodiments the modular synthetic hydrogels used in the methods described herein act not only as a biomimetic scaffold for embedded cells but as a carrier for release, e.g., controlled release, of growth factors, antibodies, cytokines, or cell penetrating peptide (CPP) fusion proteins.

Accordingly, in some embodiments, the synthetic tissue to be administered also includes one or more exogenous growth factors, antibodies or cell penetrating peptide (CPP) fusion proteins. Growth factors are known in the art to include growth factors or growth factor-like molecules, or molecules that induce differentiation/de-differentiation.

Suitable exogenous growth factors for inclusion in the above-mentioned synthetic tissue include, but are not limited to, one or more of BDNF, VEGF, IGF1, bFGF/FGF2, Angl, Ang 2, BMP 2, BMP 3a, BMP 3b, BMP 4, BMP 5, BMP 6, BMP 7 (OP-1), CTNF, EGF, EPO, aFGF/FGF1, bFGF/FGF2, G-CSF, GDF10, GDF15, GDNF, GH, GM-CSF, HB-EGF, LIF, NGF, NT-3, NT 4/5, Osteopontin, PDGFaa, PDGFbb, PDGFab, P1GF, SCF,

SDF1/CXCL12, and TGFβ. Suitable antibodies include, but are not limited to, antibodies against any of α-synuclein, Aβ oligomers, Tau oligomers, fibrin, and toll-like receptor (TLR) 4. Suitable cytokines include, but are not limited to, cytokines shown to suppress inflammatory response in the brain, e.g., IL-10, IL-4, IL-6, IL-11, IL-1alpha, IL-1beta, IL-18 and IL-13.

In some embodiments the synthetic tissue includes a CPP-transcription factor fusion protein. Of particular interest are CPP-transcription factors that can convert cells from one lineage into another, e.g., glial cells into neurons. See, e.g., Hu et al. (2014), Xu et al. (2017). Suitable CPP transcription factors include CPP fusions of one or more of, Ascl 1, Brn2, Nurr1, Foxa2, Ngn2, Lmx1a, Pitx3, and Otx2. Various CPPs suitable for use in generating CPP fusion proteins are known in the art as described in, e.g., Peraro et al. (2018) and Kaitsuka et al. (2015).

In other embodiments the synthetic tissue also includes one or more types of exogenous exosomes. Examples of suitable types of exogenous exosomes for treatment of a variety of neurological conditions have been exemplified in the art. See, e.g., Xia et al. (2019).

### Generation of Modular Synthetic Hydrogels

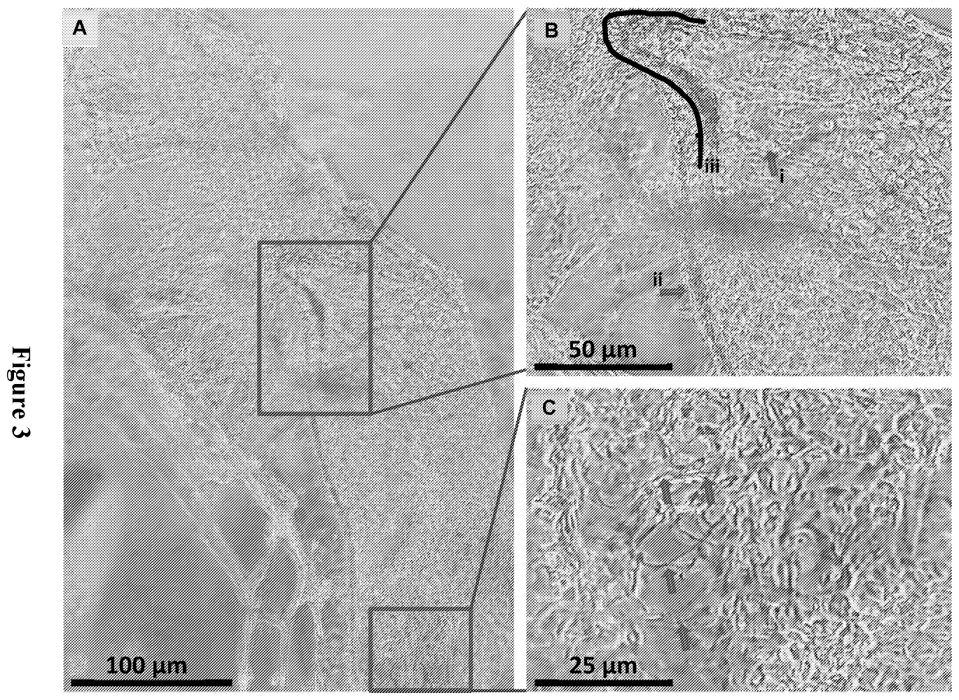

Hydrogel precursor molecules, such as conjugated cysteine terminated star-PEG and heparin-maleimide conjugates as illustrated in FIG. 3, are dissolved in PBS or other appropriate solution/buffer and stored at a low temperature or on ice. The cooled solutions are combined and gently mixed to allow gel formation to proceed. Gel formation may occur within a few seconds to a couple of minutes. The concentrations of the precursor starting solutions can be adjusted as required to maintain a desirable solid content. The starting solutions, mixing and subsequent processing/distribution steps may be archived by manual, semi-automatic or fully automated procedures. It will be appreciated that robots are useful, for example, for accurately running multiple steps and with very small volumes, for spray polymerisation and 3D printing applications, and to maintain solution/gel sterility. In particular, reactions may be performed using liquid handling robot instrumentation able, for example, to dispense, combine, and mix and/or distribute discrete volumes of starting materials. As described herein additional materials include cells and biologically active molecules such as growth factors, antibodies, small molecules, cell penetrating peptides etc. Cell viability is maintained by ensuring optimal conditions for cell viability as known in the art. Factors routinely considered in this regard include pH, temperature, gas-exchange factors, concentration and the specific media employed.

Cell populations may be cultured after isolation for a time and under conditions selected depending upon the desired maturation state. For example, cells may be cultured for less than 24 hours, or for up to about 4-6 months. In some embodiments the cells are not cultured prior to micro-bead manufacture. Mixtures of different cells types or cells types at different stages of maturation/differentiation may be cultured together or separately. Once the synthetic tissues are formed, further culture/maturation protocols can be performed, depending upon the application.

In one embodiment, to allow synthetic tissue comprising cell populations to be administered without damaging the delicate tissue that has formed, they are manufactured in very small volumes. Synthetic tissue can be generate in the form of microbeads/micro-particles, including microparticles of about 0.2 μl volume. Individual microparticles may be cultured separately or together with other microparticles of the same or different composition/shape/size.

For example, microparticles comprising cell populations may be cast directly at the bottom of a tissue culture plate or on other suitable surface.

### EXAMPLES

### Example 1—Preparation and use of Synthetic Brain Micro-tissues (SBMs) for the treatment of Parkinson's Disease

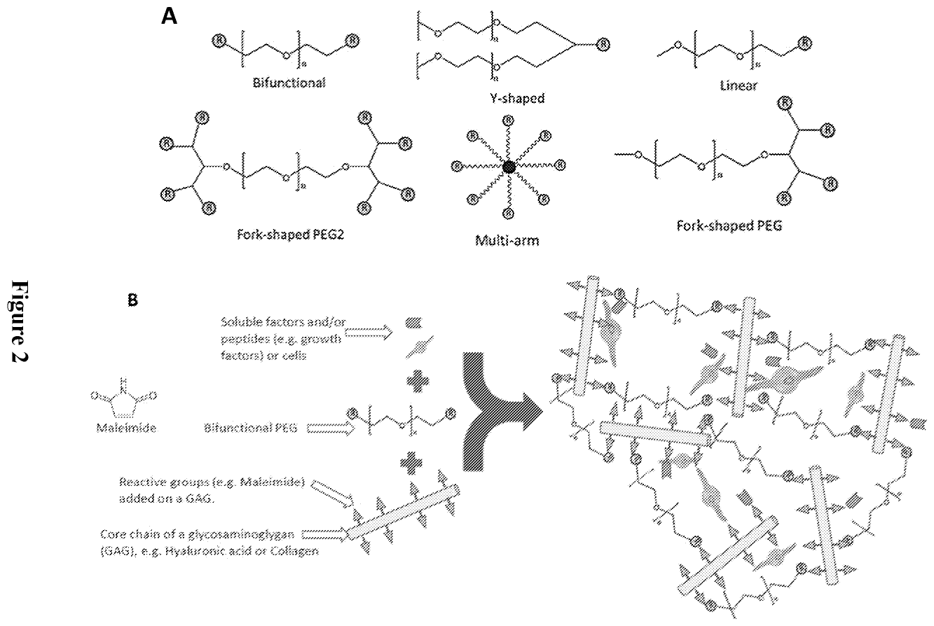

Human fetal astrocytes and mid brain dopaminergic neurons are cultured after isolation. Prior to synthetic brain microtissue preparation, the cultured cells are treated with Accutase® (Invitrogen) according to the manufacture's instructions to obtain a single cell suspension, and collected in cell separation medium, Neurobasal™ Medium, and centrifuged for 10 min at 160-180 (170)×g. The cells are resuspended at a concentration of 8×10⁶ cells per ml in phosphate buffered saline (PBS) to obtain a cell-PBS solution. The cell-PBS solution is then mixed with a PBS-Heparin solution at 1:1 (v/v) ratio (“Cell-HEP” solution) and optionally additional components, (eg cytokines, growth factors, small molecules in concentration between 0.01 nM-10000 mM etc). Heparin molecules in the PBS-Heparin solution are functionalized with six maleimide groups (HEP-HM6) having a molecular weight of 15,000 g/mol, as described in Tsurkan et al. (2013) and illustrated schematically in FIG. 2. A cross-linking solution, is obtained by dissolving four-arm (“starPEG”) functionalized with enzymatically cleavable peptide sequences on each arm having (total molecular weight of 15,500 g/mol) in PBS (“StarPEG conjugate solution”). Thiol-containing cysteine residues within the starPEG-linked peptides are available for reaction through Michaels addition with maleimide groups on the HEP-HM6 conjugate. Hydrogel crosslinking is initiated by mixing the Cell-HEP-HM6 solution with the StarPEG conjugate solution. The hydrogel matrix components (StarPEG conjugate and HEP-HM6) are combined at a molar ratio between about 0.75 to 1 starPEG-conjugate:HEP-HM6 (corresponding to a cross-linking degree of 0.75).

In the resulting microtissue that is formed, the total content of solid materials should be about 4% (excluding the cells). Based on the above-described protocol, a 0.2 μl (0.2 microliter) volume microtissue is formed according to the following steps:
1. IPs derived human midbrain dopaminergic neurons and human iPS derived astrocytes in a ratio 30:70 are resuspended at 2×10⁶/ml in PBS.
2. 0.0448 mg of HEP-HM6 are dissolved in 5 μl of PBS.
3. 0.0347 mg StarPEG-MMP conjugate dissolved in 10 μl of PBS.
4. The solutions are mixed together and approximately 20,000×0.2 μl drops are rapidly arrayed using a liquid handling robot onto a hydrophobic surface (e.g., parafilm). Hydrogel formation is completed in approximately 40 secs.
5. The hydrogel microbeads are placed individually into 384-well culture plates, where each well contains 20 μl of culture medium (ScienCell Research Laboratories, catalog number 1801), supplemented with 1% of growth factors medium (SRL, catalog number 1852) and 1% of penicillin/streptomycin solution (SRL, catalog number 0503)). Cells are fed/media changed every other day.
6. The microbeads are cultured for 21 days at 37° C., 5% CO₂ 95% air.
7. After the culture period, and progressive, cell-mediated breakdown of hydrogel synthetic matrix, the resulting “synthetic brain microtissue beads” (“SBMs”) develop a number of distinctive characteristics: a composition of about 70% (by weight) of cell secreted extracellular matrix; a stiffness between about 100 Pa to about 700 Pa; the presence of more than 200 networks, each of which includes at least three interconnected neurons, and has more than five branches (connections with other networks).
8. The SBMs are pooled and mixed at 1:1 (v/v) ratio with a solution of starPEG-MMP conjugate and HEP-HM6, which forms a secondary synthetic hydrogel layer having a cross-linking ratio=0.2 in a total volume of 20 μl (10 μl of SBMs and 10 μl of starPEG-MMP-REP-HM6 solution). The secondary hydrogel layer assists in localizing the component SBMs once introduced into the tissue, and also provides a suitable interface substrate for connectivity and interactions to develop between cells in the transplanted SBMs and cells in the surrounding tissue so as to promote long term functional integration of SBMs into the host brain.
9. The pooled SBM-secondary hydrogel is then loaded into a reservoir of a medical device (e.g., a microsyringe) and approximately 2 μl of the SBM-hydrogel are delivered by stereotaxic administration into the substantia nigra of an anesthetized (6-hydroxydopamine (6-OHDA) rat at a rate of about 0.2 μl/minute (Duty and Jenner, 2011).

### Experimental Plan and Specific Endpoints to be Assessed

TABLE 1

| Proposed experiments | Expected Results |
|---|---|
| Implantation of products in animal models: | Implanted cells are alive and produce DOPA/GABA |
| 4 time-points (animal sacrifice) | No signs for uncontrolled proliferation |
| 4 concentrations of n/a | No signs of unwanted cell differentiation |
| 3 implantation stages | No signs of tumour formation |
| 4 replicates | Number of implanted neurons is similar in all time- |
| → ≈200 rats/indication | points |
| Evaluation: | Implanted cells are restricted in the area of interest |
| Integration and survival of implants | Native cells have migrated in the implants |
| Potential cell migration | Synaptic connectivity between implanted and native |
| Infiltration of cells in capillaries | cells |
| Production of DOPA/GABA | Mice have alleviated phenotypical symptoms |
| Synaptic connectivity | (e.g. tremor, spasms) |
| Phenotypical examinations | |
| (e.g. maze, tremor and other tests) | |

### Example 2—Preparation and use of Synthetic Brain Microtissues (SBMs) for the Treatment of Stroke

Human fetal astrocytes are cultured after isolation. Prior to synthetic brain microtissue preparation, the cultured cells are treated with Accutase° (Invitrogen) according to the manufacturer's instructions to obtain a single cell suspension, and collected in cell separation medium, Neurobasal™ Medium, and centrifuged for 10 min at 160-180×g. The cells are resuspended at a concentration of 8×10⁶ cells per ml in phosphate buffered saline (PBS) to obtain a cell-PBS solution. The cell-PBS solution is then mixed with a PBS-Hyaluronic acid (HAL) solution at a 1:1 (v/v) ratio (“Cell-HAL” solution) and optionally additional components, (e.g. cytokines, growth factors, or small molecules). HAL molecules in the PBS-HAL solution are functionalized with eight maleimide groups (HAL-HM8) having a molecular weight of 17,000 g/mol. A cross-linking solution, is obtained by dissolving linear PEG (“LPEG”) functionalized with enzymatically cleavable peptide sequences on each end having (total molecular weight of 17,000 g/mol) in PBS (“LPEG conjugate solution”). Thiol-containing cysteine residues within the PEG-linked peptides are available for reaction through Michaels addition with maleimide groups on the HAL-HM8 conjugate. Hydrogel crosslinking is initiated by mixing the Cell-HAL-HM8 solution with the PEG. The hydrogel matrix components (LPEG conjugate and HAL-HM8) are combined at a molar ratio between about 1 to 2 PEG-conjugate(s): HAL-HM8.

In the resulting microtissue that is formed, the total content of solid materials should be about 4% (excluding the cells). Based on the above-described protocol, a 0.2 μl volume microtissue is formed according to the following steps:
1. hiPSC-derived human neural progenitor cells (NPCs) and hiPSC-derived astrocytes in a ratio 30:70 are resuspended at 2×10⁶/ml in PBS.
2. 0.5 mg of HAL-HM8 are dissolved in 10 μl of PBS.
3. 0.5 mg LPEG-MMP conjugate dissolved in 10 μl of PBS.
4. The solutions are mixed together and approximately 20,000×0.2 μl drops are rapidly arrayed using a liquid handling robot onto a hydrophobic surface (e.g., parafilm). Hydrogel formation is completed in approximately 40 secs.
5. The hydrogel microbeads are placed individually into 384-well culture plates, where each well contains 20 μl of culture medium (SRL, catalog number 1801), supplemented with 1% of growth factors medium (SRL, catalog number 1852) and 1% of penicillin/streptomycin solution (SRL, catalog number 0503)). Cells are fed/media changed every other day.
6. The microbeads are cultured for 30 days at 37° C., 5% CO₂ 95% air.
7. After the culture period, and progressive, cell-mediated breakdown of the hydrogel synthetic matrix, the resulting “synthetic brain microtissue beads” (“SBMs”) develop a number of distinctive characteristics: a composition of about 70% (by weight) of cell secreted extracellular matrix; a stiffness between about 100 Pa to about 3000 Pa; the presence of more than 200 networks, each of which has more than five branches (connections with other networks); and more than 12,000 distinct neural projections.
8. The SBMs are pooled and mixed at 1:1 (v/v) ratio with a solution of LPEG-MMP conjugate and HAL-HM8, which forms a secondary synthetic hydrogel layer having a cross-linking ratio=0.2 in a total volume of 20 μl (10 μl of SBMs and 10 μl of LPEG-MMP-HAL-HM8 solution). The secondary hydrogel layer assists in localizing the component SBMs once introduced into a tissue in vivo, and also provides a suitable interface substrate to establish connectivity and interactions between cells in the transplanted SBMs and host cells in the surrounding tissue so as to promote long term functional integration of SBMs into the host brain.
9. The pooled SBM-secondary hydrogel is then loaded into a reservoir of a medical injection device (e.g., a microsyringe) and approximately 2 μl of the SBM-hydrogel are delivered by stereotaxic administration into the cortex of an anesthetized mouse that in which an ischemic stroke was induced, e.g., by as described in the photothrombotic stroke model of (Yu et al., 2015).
10. The SBM-hydrogel is injected in a necrotic brain area generated around a photo-induced ischemic blood clot.
   Summary Table of Experimental plan and Specific Endpoints to be Assessed

TABLE 2
Summary of Specific Experimental Endpoints to be Assessed

| Proposed Experiments | Expected Results |
|---|---|
| Implantation of products in animal models: | Implanted cells are alive and produce VEGF and/or |
| 4 time-points (animal sacrifice) | BDNF and/of FGF |
| 4 concentrations of | No signs of uncontrolled proliferation |
| (neurons/astrocytes) | No signs of unwanted cell differentiation |
| 3 implantation stages | No signs of tumour formation |
| 4 replicates | Number of implanted neurons is similar at all time- |
| → ≈200 rats/indication | points |
| Evaluation: | Implanted cells are localised to the area of interest |
| Integration and survival of implants | Native cells have migrated into the implants |
| Potential cell migration | Synaptic connectivity is evident between implanted |
| Infiltration of cells in capillaries | and native cells |
| Production of VEGF and/or BDNF | Mice exhibit reduced behavioural symptoms (e.g. |
| and/of FGF | tremor, spasms) |
| Synaptic connectivity in lesion area | |
| Behavioural assessment (e.g., maze | |
| navigation, tremor, and rotorod) | |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU2018904068 filed 26 Oct. 2018, the entire contents of which are incorporated herein by reference.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Blesa et al. (2014), Frontiers in Neuroanatomy, 8(155).
Derakhshanfar et al. (2018), Bioactive Materials, 3(2):144-156.
Duty and Jenner (2011), Br. J. Pharmacol. 164(4), 2011.
Gotz et al. (2018), Nature Reviews Neuroscience, 19(10):583-598.
Hadjigeorgiou et al. (2017), J Neurosurgical Sciences, 61(6):652-664.
Hu et al. (2014) Stem Cells Translational Medicine, 3(12):1526-1534.
Katisuka et al. (2015), Int J Mol Sci, 16(11):26667-26676.
Lemke et al. (2017), Current Opinion in Biotechnology, 47:36-42.
McCabe et al. (2018), Neuropharmacology, 134(Pt B): 169-177.
Peraro et al. (2018), Agnew Chem Int Ed Engl., 57(37):11868-1181.
Santos et al. (2017), Stem Cell Reports, 8(6):1757-1769
Shi et al. (2012), Nature Protocols, 7(10):1836-1846.
Studer et al. (2012), Progress in Brain Research, 200:243-263.
Tsurkan et al. (2013), Advanced Materials, 25(18):2606-2610.
Xia et al. (2019), Progress in Neurobiology (in press).
Xu et al. (2017), Redox Biology, 11:606-617.
Yu et al. (2015), J Neurosci Methods, 239:100-7.

1. (canceled)

2. A method for treating a neurological disorder in a subject in need thereof, comprising administering to the subject a therapeutically effective amount of a synthetic tissue comprising a cell population comprising (a) one or more nervous system cell types, or (b) multipotent cells;
wherein the cell population is embedded within a modular synthetic hydrogel that is biocompatible.

3. (canceled)

4. The method according to claim 2, wherein the subject is suffering from a neurological disorder selected from the group consisting of: Alzheimer's Disease, vascular dementia, Parkinson's Disease, Huntington's Disease, stroke, ischemic stroke, haemorrhagic stroke, optic nerve disease, spinal cord injury, peripheral nerve injury, demyelinating disease, and traumatic brain injury.

5-6. (canceled)

7. The method according to claim 2, wherein the nervous system cell types are selected from the group consisting of neurons, neural progenitor cells, glial cells, and any combination thereof.

8. The method according to claim 2, wherein the cell population comprises:
(i) neurons;
(ii) glial cells;
(iii) neurons and glial cells;
(iv) neurons and astrocytes;
(v) neurons and microglia;
(vi) neurons and oligodencrocytes,
(vii) neurons, astrocytes, and microglia, or
(viii) neurons, astrocytes, and oligodendrocytes.

9. The method according to claim 8, wherein the neurons are selected from the group consisting of monoaminergic neurons, catecholaminergic neurons, glutamatergic excitatory neurons, GABAergic inhibitory neurons, motor neurons, cholinergic neurons, and any combination thereof

10-16. (canceled)

17. The method according to claim 9, wherein neurons in the cell population prior to the administration exhibit at least one functional characteristic associated with neuronal maturation selected from the group consisting of:
secretion of a cognate neurotransmitter, secretion of a growth factor, expression of a mature neuronal protein marker, surface expression or subcellular localisation of a neurotransmitter receptor, intrinsic electrical activity, and synaptic connectivity.

18-28. (canceled)

29. The method according to claim 2, wherein the modular synthetic hydrogel comprises one or more hydrogel subunit materials selected from the group consisting of polyethylene glycol (PEG), hyaluronan, gelatin, thiol-modified hyaluronan, acrylated hyaluronic acid thiol-modified chondroitin sulfate, thiol-modified gelatin, acrylic copolymers, polyvinylidene fluoride, chitosan, polyurethane isocyanates, polyalginate, cellulose acetate, polysulfone, polyvinyl alcohols, and polyacrylonitrile.

30. (canceled)

31. The method according to claim 29, wherein the modular synthetic hydrogel further comprises one or more peptides or polypeptides linked to the one or more hydrogel subunit materials.

32. The method according to claim 31, wherein the one or more peptides or polypeptides comprise at least a first and a second peptide or polypeptide.

33. The method according to claim 31, wherein at least one of the one or more linked peptides or polypeptides is enzymatically cleavable.

34-35. (canceled)

36. The method according to claim 31, wherein at least one of the one or more linked peptides comprises a convertible functional group.

37-41. (canceled)

42. The method according to claim 2, wherein the modular synthetic hydrogel comprises PEG and at least one of heparin, hyaluronan, chitosan, gelatin, chondroitin sulphate, and collagen.

43-57. (canceled)

58. The method according to claim 2, wherein the synthetic tissue is implanted into or proximal to the brain, spinal cord, optic nerve, or a peripheral nerve of the subject.

59-60. (canceled)

61. The method according to claim 2, wherein the synthetic tissue is provided in the form of microparticles.

62. The method according to claim 61, wherein the microparticles have a diameter of about 1 μm to about 2000 μm or about 50 μm to about 500 μm.

63. (canceled)

64. The method according to claim 61, wherein the microparticles comprise at least first and second populations of microparticles that differ from each other in at least one of the following characteristics: cell types, proportions of cell types, spatial distribution of cell types, hydrogel subunit materials, linked peptides or polypeptides, exogenous growth factors.

65. (canceled)

66. The method according to claim 61, wherein the synthetic tissue is administered as an aqueous suspension having a viscosity greater than 100 Pa.

67-70. (canceled)

71. The method according to claim 61, wherein the synthetic tissue is injected into or proximal to the brain, spinal cord, optic nerve, or a peripheral nerve of the subject.

72-75. (canceled)

76. A synthetic tissue for use in treating a neurological disorder or repairing nervous system tissue, wherein the synthetic tissue comprises a cell population comprising (a) one or more nervous system cell types, or (b) multipotent cells, and wherein the cell population is embedded within a modular synthetic hydrogel that is biocompatible.

77-81. (canceled)

82. The synthetic tissue according to claim 76, wherein the synthetic tissue is provided in the form of microparticles.

83. (canceled)


SEQUENCE LISTING (3 sequences)

SEQ ID NO: 1 - protein, 12 aa, Artificial Sequence
  Metalloprotease cleavage site 1
GPQGIWGQGGCG

SEQ ID NO: 2 - protein, 10 aa, Artificial Sequence
  Metalloprotease cleavage site 2
SIKVAVGWCG

SEQ ID NO: 3 - protein, 8 aa, Artificial Sequence
  Metalloprotease cleavage site 3
YIGSRGCG